(19) United States
(12) Patent Application Publication
(10) Pub. No.: US 2008/0194689 A1
(43) Pub. Date: Aug. 14, 2008

(54) Disinfectant and Germicidal Agent

(76) Inventor: Sven Reichwagen, Bromskirchen (DE)

Correspondence Address: PETERS VERNY , L.L.P., 425 SHERMAN AVENUE, SUITE 230, PALO ALTO, CA, 94306, US

(21) Appl. No.: 11/915,249

(22) PCT Filed: May 26, 2006
(86) PCT No.: PCT/EP2006/005016

§ 371 Date: Feb. 26, 2008

(30) Foreign Application Priority Data
May 25, 2005 (DE) 102005024001.1

(51) Int. Cl. A01N 37/00; A61L 2/18; A01P 1/00; A01P 7/04; A01P 15/00

(52) U.S. Cl. 514/574; 422/28

(57) ABSTRACT

The invention relates to a composition containing at least one percarboxylic acid and at least one ethoxylated, non-ethoxylated or oxypropylene units containing sorbitan ester. Moreover, the invention also relates to a method for producing such a composition and the use thereof.

The inventive percarboxylic acid composition especially serves as a disinfectant and germicidal agent. Surprisingly, the application of sorbitan esters causes percarboxylic acid to be significantly stabilized and in this way enables the inventive products to be utilized longer than the percarboxylic acid compositions known from prior art.

The invention relates to a composition containing at least one percarboxylic acid. The invention moreover relates to a method for producing such a composition and the use thereof.

Basically, the use of percarboxylic acids as bactericide for disinfection purposes is known from prior art, as for example disclosed by the European patent specification EP 0 677 990 B1 wherein particularly the use of peracetic acid has been described. The disinfectant is suited, inter alia, for the treatment of medical equipment, for instance endoscopes.

Percarboxylic acid solutions are produced by converting carboxylic acid with hydrogen peroxide in an aqueous medium in the presence of an acidic catalyst, usually sulfuric acid. The reaction that takes place results in an equilibrium forming between the percarboxylic acid as reaction product on the one hand and the reactants, i.e. the carboxylic acid and hydrogen peroxide on the other. After the reaction has been completed the reaction mixture is often diluted further through the application of a solvent to bring about the desired percarboxylic acid concentration. For purposes such as cleaning of toilets solutions may for example be employed that contain 0.5 to 1% w/w of peracetic acid. Normally, the percarboxylic acid is produced initially in the form of a concentrated solution because the time period required to adjust the equilibrium would otherwise be excessive.

Even if a concentrated equilibrium solution of the percarboxylic acid is diluted with water the system equilibrium point changes in such a manner that the original reactants are regenerated; and such an equilibrium adjustment may as well require a long period of time to complete, i.e. in the range of several weeks or even months. Accordingly, the diluted percarboxylic solution is a solution of variable composition the effectiveness of which lessens more and more as the percarboxylic acid concentration diminishes. Another problem arises in that the final composition to some extent contains further constituents weakening the stability of the percarboxylic acid even more. For that reason, EP 0 677 990 B1 suggests a solution providing for the actually employed disinfectant to be prepared immediately before its application using two solutions of which one consists a peracetic acid and the second of another component, for instance a corrosion inhibitor reducing the stability of the peracetic acid. After the disinfectant solution has been prepared it must be put to use quickly, that is at a time before the equilibrium has been re-established, i.e. before the percarboxylic acid concentration has not again been reduced to a significant extent.

Basically, diluting the percarboxylic acid solution just before it is put to use is an option conducive to prolonging the useful life of the percarboxylic acid but has the disadvantage that its application is quite complex and requires greater effort. For example, the end user who wishes to employ the disinfectant let us say in sanitary facilities must first mix two solutions with each other that are stored in two different containers and for this purpose must use a third container before he or she is in a position to apply the solution. As it is frequently desirable especially in sanitary facilities to use the solution in poorly accessible spaces, for example inside the toilet, applying it by means of a spray bottle, the prepared solution would have to be filled additionally into such a spray bottle which would render its handling and use even more cumbersome. As is commonly known, end users are accustomed to employing solutions that are ready for use without further manipulation.

In this context another problem is encountered in that a highly concentrated peracetic acid solution must initially be diluted and therefore has to be handled with greatest care because when coming into contact with the skin the solution would cause chemical burns. Particularly problematic, of course, is a contact with mucous membranes: another aggravating issue adding to the problems that at least the ultimate consumer inexperienced in handling chemicals faces when using percarboxylic acid solutions that must first be diluted before they can be applied.

Proceeding from what is known from prior art as described hereinbefore it is therefore the objective to provide a composition containing at least one percarboxylic acid that has sufficient stability so that the rather cumbersome method of dilution shortly before application as described above can be dispensed with.

According to the invention this objective is reached by providing a composition that contains aside from at least one percarboxylic acid, furthermore at least one ethoxylated, non-ethoxylated or oxypropylene units containing sorbitan ester.

The invention is based on findings according to which the addition of a significant amount of a sorbitan ester considerably increases the stability of the percarboxylic acid. Initial tests have shown that the percarboxylic acid concentration virtually remains unchanged over a time span of several weeks so that for the composition according to the invention a minimum usability period of two years can be expected. This effect is thought to be caused by the sorbitan ester partially complexing the percarboxylic acid and reducing the movability of the atomic oxygen initially arising when the percarboxylic acid decomposes to such an extent that the probability of a reverse reaction increases considerably.

The sorbitan esters involved are in particular ethoxylated sorbitan esters, so-called polysorbates. These are sold inter alia under the tradename of “Tween®” by the company of ICI. In particular, polysorbate 20, 21, 40, 60, 61, 65, 80, 81 or 85 (Tween 20, 21 etc.) can be used. These are polyethoxysorbitan laurates, -palmitates, -stearates, -tristearates, -oleates and -trioleoates. Usually, these polysorbates are oily liquids well soluble and dispersible in water. Basically, non-ethoxylated sorbitan esters, i.e. mono-, di- or tri-esters of sorbitans with fatty acids may also be used. Expediently, the concentration of the sorbitan esters in the composition amounts to 1 to 50% w/w, in particular>15% w/w. Especially preferred is a concentration ranging between 16 and 30% w/w and most particular between 18 and 25% w/w. An overview of polysorbates that may be used can be seen from the following table.

| | |
|---|---|
| Polysorbate 20 | Polyoxyethylene (20) sorbitan monolaurate |
| Polysorbate 21 | Polyoxyethylene (4) sorbitan monolaurate |
| Polysorbate 40 | Polyoxyethylene (20) sorbitan monopalmitate |
| Polysorbate 60 | Polyoxyethylene (20) sorbitan monostearate |
| Polysorbate 61 | Polyoxyethylene (4) sorbitan monostearate |
| Polysorbate 65 | Polyoxyethylene (20) sorbitan tristearate |
| Polysorbate 80 | Polyoxyethylene (20) sorbitan monooleate |
| Polysorbate 81 | Polyoxyethylene (5) sorbitan monooleate |
| Polysorbate 85 | Polyoxyethylene (20) sorbitan trioleate |

The use of polysorbate 20 (Tween 20) has proven particularly favorable.

Depending on the relevant purpose, the percarboxylic acid concentration of the composition may range between 0.1 and 45% w/w, especially preferred is a concentration ranging between 0.1 and 20% w/w and in particular between 5 and 15% w/w. Naturally, the higher the percarboxylic acid concentration the higher the effectiveness of the composition; for many purposes and applications, however, it will be sufficient to use concentrations of up to 1% w/w. As a rule, a concentrate is prepared initially that has a percarboxylic acid concentration in the range of between 10 and 20% w/w but said concentration will subsequently be reduced through dilution using a solvent. However, other than is known from the above described prior art said dilution may be effected long before the composition is put to use because the percarboxylic acid in the diluted composition also has sufficient stability.

It has proven particularly advantageous to select compositions that contain at least two percarboxylic acids because experience has shown that this may often produce synergistic effects. It was thus found that different kinds of percarboxylic acid were particularly suited for numerous specific purposes. For example, a number of toxins, both mykotoxins and endotoxins, having an aldehyde or ketonic function are effectively destroyed by means of perbenzoic acid via a Baeyer-Villiger oxidation. For instance, perbenzoic acid can be effectively used to combat aflatoxins produced by fungus which are both toxic and carcinogenic. Moreover, perbenzoic acid acts as a catalase inhibitor. On the other hand, peracetic acid is suited to effectively attack pore proteins of bacteria whereas percitric acid has proven especially suited to decompose biofilms and calcium encrustation in biofilms. Persorbic acid is capable of entering particularly effectively lipid layers and in this manner penetrates through cell membranes in order to deactivate bacteria for example.

Basically, it can be said that well-soluble peracids cause rapid effects (for example pertartaric acid, peracetic acid or percitric acid) whereas slightly soluble peracids have a more lasting effect (for example persorbic acid or perbenzoic acid). Persuccinic acid advantageously has additional preserving properties. To suit the respective application several percarboxylic acids may be combined with each other to enable bacteria, viruses, toxins etc. to be attacked as effectively as possible.

Especially preferred, either individually or in combination with another percarboxylic acid, is the use of perbenzoic acid, percitric acid, peracetic acid, persorbic acid, permalonic acid, permaleic acid, perfumaric acid, pertartaric acid and/or persuccinic acid. However, almost any desired percarboxylic acid with carbon chains of optional length may be employed. However, due to the fact that particularly long-chained carboxylic acids have relatively poor water solubility it is preferred to use percarboxylic acids having a carbon chain not exceeding C₈.

Especially suited combinations are compositions that contain both perbenzoic acid and persorbic acid and, if though expedient, additionally pertartaric acid or persuccinic acid. Combinations of percitric acid and perbenzoic acid are suited for the dissolution of biofilms.

To further slow down the reverse reaction of percarboxylic acid to form the underlying percarboxylic acid the composition as a rule contains an inorganic peroxide, in particular hydrogen peroxide, capable of oxidizing the carboxylic acid to form percarboxylic acid. In this manner the equilibrium is shifted towards the percarboxylic acid. Aside from this, the hydrogen peroxide itself present in the composition may as well act as a disinfectant additionally to the percarboxylic acid. The concentration of the inorganic peroxide preferably comes to 20 to 40, especially 30 to 40% w/w.

Moreover, the composition usually also contains the carboxylic acid corresponding to and forming the basis of the percarboxylic acid. On the one hand, the carboxylic acid is usually present in the composition alone for the reason that an equilibrium establishes between carboxylic acid and percarboxylic acid; however, this equilibrium, on the other hand, may additionally be shifted towards percarboxylic acid in that an excess of carboxylic acid is added to the composition. An equilibrium forms with approx. 50% of the carboxylic acid being present usually in the form of percarboxylic acid.

The composition may comprise customary additives, in particular solvents, surfactants, sequestrants, pH regulators, corrosion inhibitors, peracid stabilizers, complexing agents, defoaming agents, coloring substances and/or scents. Especially expedient is the addition of surfactants if the composition is meant to serve as cleaning agent as well. A suitable surfactant is, for example, SDS (sodium dodecyl sulfate). Corrosion inhibitors are especially useful if the composition is to be used for the treatment of metal surfaces. As corrosion inhibitors alkali-metal phosphates, preferably potassium phosphates, may be employed. Especially preferred is the use of dipotassium hydrogen orthophosphate (K₂HPO₄). The concentration of the corrosion inhibitor is preferably in the range of between 0.1 and 5% w/w.

Furthermore, the solution may comprise a peracid stabilizer, preferably of a concentration ranging between 0.1 and approx. 2% w/w. As peracid stabilizers phosphonic acid and its salts or dipicolinic acid may be employed.

Minor amounts of phosphoric acid may also be used with said phosphoric acid, especially as acidic catalyst, serving to provide assistance to a Baeyer-Villiger oxidation process. Conceivable is also the use of one or several oxidation-resistant permeation agents such as DMSO.

Primarily, water or an alcohol shall serve as solvent, especially ethanol or isopropanol. Alcohols offer advantages in that they have additional bactericidal and fungicidal properties so that the germicidal effects of the inventive composition are even enhanced.

Basically, the composition may be prepared as a solution, solid or gel; but if formulated as a solid substance it must usually be incorporated into a solvent before it is applied. In case the composition is to be prepared in the form of a gel at least one gelatinizer is added to the composition solution, preferably Aerosil, alginate, agarose or at least one acrylate. The resulting gel may then be applied to a surface which is to be treated.

When prepared in the form of a solid substance the solvent is omitted and the respective components are furnished as powdery blend, as granules or in tablet form.

The pH value of the composition should be in the acidic range so that the percarboxylic acids are present as free acids and not as salts because percarboxylic acid decomposes relatively quickly in the alkaline range. Typically, the pH value of an initially prepared concentrate of the composition lies between 0 and 2, whereas the pH value of the solution diluted for application purposes ranges between 3 and 6. If necessary, the pH value may be adjusted by making use of pH regulators, preferably by adding acids, bases and/or buffers.

Aside from the inventive composition itself the invention also relates to a method for producing such a composition. According to the inventive method a carboxylic acid or a carboxylic acid derivative is converted with inorganic peroxide, in particular hydrogen peroxide, in the presence of an ethoxylated, non-ethoxylated or oxypropylene units containing sorbitan ester. Carboxylic acid derivatives can be employed that after conversion yield percarboxylic acids, especially carboxylic acid anhydrides, -amides or -esters.

Typically, the reaction takes place at temperatures<50° C., in particular between 10 and 30° C. Preferably, the reaction is to be effected at room temperature, i.e. between 20 and 25° C.

In the reaction, sorbitan ester may directly serve as solvent for the carboxylic acid/carboxylic acid derivative.

Through the absence of water the equilibrium of the reaction of the carboxylic acid with hydrogen peroxide to form percarboxylic acid and water is shifted to the product side. This effect may even be increased by using carboxylic acid anhydrides instead of the carboxylic acid proper because these additionally will remove water from the reaction mixture. The presence of sorbitan ester, in particular the polysorbate, causes the percarboxylic acid to be significantly stabilized so that a relatively high percarboxylic acid content forms in the reaction mixture. Such a percarboxylic acid stabilization can still be observed even when upon completion of the reaction (i.e. after the equilibrium has been attained) the reaction mixture is finally diluted with the aid of a suitable solvent, usually water or alcohol.

However, the conversion may also take place directly in an aqueous solution which ensures that altogether a homogenous composition is produced. However, to be able to adequately shift the reaction equilibrium initially towards the percarboxylic acid side it is thought expedient to aim for a relatively highly concentrated conversion process first and then bring about the dilution by means of a solvent, particularly water.

During the conversion sorbitan ester is preferably used at a concentration ranging between 16 and 30% w/w, especially between 18 and 25% w/w. The inorganic peroxide, i.e. in particular hydrogen peroxide, is expediently used at a concentration of between 20 and 40% w/w, especially between 30 and 40% w/w.

The conversion process yielding percarboxylic acid may further be accelerated by adding an acidic catalyst. This may be (concentrated) sulfuric acid, phosphoric acid or methanesulfonic acid. Sulfuric acid offers advantages in that it moreover has a dehydrating effect. Basically however, other highly acidic, inorganic mineral acids may be employed as well.

The time required for the conversion to complete typically ranges between one day and six weeks because the equilibrium adjustment is relatively slow. It has turned out that a period of between two and four weeks is particularly suited.

Expediently, an excess of inorganic peroxide, i.e. hydrogen peroxide as a rule, is applied in the reaction compared to the carboxylic acid or the carboxylic acid anhydride. In this manner the equilibrium can be shifted towards the product side and it is ensured, moreover, that carboxylic acid arising due to decomposition is re-oxidized to form percarboxylic acid.

Dilution by means of a solvent is typically effected at a ratio of between 1:2 and 1:50, especially at a ratio of between 1:5 and 1:20.

The composition in accordance with the invention may be employed for numerous applications aimed at combating detrimental microorganisms and the metabolic products they produce, viruses etc. For example, the composition thus serves especially as disinfectant, crop protection agent, plant fortifier and germicidal agent as well as bactericide, fungicide, insecticide or pesticide. Moreover, the compositions in accordance with the invention may be used for the production of drugs aimed at treating bacterial or viral infectious diseases, mycoses, pediculoses or to counteract parasitic infestation.

In the process of combating bacteria, germs, fungi etc. the liberation of oxygen radicals cause the outer envelope of the cell/the cell membrane to oxidize quickly such that the outer envelope breaks and becomes destroyed with the interior of the cell exiting. Also enzymes and other proteins as well as nucleic acids are attacked by percarboxylic acids and damaged in this manner. Due to the metabolic path (energy metabolism, protein biosynthesis) being blocked as a result of this the death of the cell occurs quickly. Aside from this, the inventive composition will also destroy biotoxins such as, for example, aflatoxins or endotoxins, mykotoxins and allergens.

The percarboxylic acids of the compositions in accordance with the invention are capable of modifying the tertiary structures of proteins such that these lose their properties. Disulfide bridges, for example, can be broken up by oxidation and thiol groups as well can be oxidized to form sulfonic acids.

The compositions according to the invention are also capable of deactivating the micororganisms' centers of metabolism by oxidation of enzymes primarily containing heavy metals such as, for example, copper. Moreover, the compositions are also capable of damaging the DNA or RNA of bacteria, fungi, their spores and viruses by oxidation of the nucleotides to such an extent that genetic information is lost.

Viruses can be deactivated by causing the capsid proteins and envelope proteins to be oxidized. This leads to a disintegration of the nucleocapsid or to a modification of the capsid surface which results in the virus to become inactive. In addition and as mentioned above, the genetic information of the virus may be affected as well.

Through oxidation and the destruction of harmful microorganisms associated with it the percarboxylic acid reconverts to carboxylic acid proper so that in the end only relatively innocuous products are produced. For example, the use of perbenzoic acid has another effect in that the developing benzoic acid serves as a preservation agent which, in particular, may be helpful in the sterilization treatment of foodstuff.

The inventive composition was tested in the following fields of application and found to be effective:

Plant/crop protection and fortification:
Fortification of trees against Phytophtora ramorum and bark beetle through spray application;
Fortification of tobacco plants to combat the tobacco mosaic virus, against aphids and root and stem rot through spray application;
Fortification of potato plants to combat late blight and tuber rot (Phytophthora infestans) and the potato beetle (Leptinotarsa decemlineata) through spray application;
Fortification of cucumber plants against downy mildew, aphids, spider mites and thrips through spray application;
Fortification of the resistance of cabbage plants against thrips, bacterial spot, Xanthomonas campestris, Alternavia brassicae. Mycosphaerella spp. Leaf spot, against insects, mites, and nematodes through spray application;
Fortification of grapevines inter alia against powdery and downey mildew (Plasmopara viticola and Oidium tuckeri), anthracnosis, botrytis, esca, eutypiosis, green rot (Penicillium expansum), pink rot, angular leaf scorch (Pseudopezicula tracheiphila), black rot (Rhizoctonia solani), black spot disease, white rot, withering, leaf rolling disease, vinegar rot, panachure, rice vinegar disease, phytoplasms, Pierce's disease, grape scale, grape phylloxera or grape berry moth through spray application. Especially in this field, the use of copper and sulfur compounds or substances in ecological winegrowing or fungicides in customary winegrowing can be dispensed with or at least reduced if this agent is applied. Especially for the combating of black rot (Guignardia bidwellii), a fungus having infested the winegrowing areas at the Moselle, Nahe river or Middle Rhine for some years now, the composition in accordance with the invention has proven highly effective.

In the field of wine production the inventive composition may as well be advantageously used for the disinfection of wine barrels and in this way replace sulfur compounds, in particular SO₂.

The compositions according to the invention may especially be used for combating Brettanomyces (Brettanomyces bruxellensis). This is a yeast strain frequently found in wine barrels causing them to be prematurely disposed of unnecessarily. As regards red wines Brettanomyces create a special problem due to the fact that the metabolic products (volatile phenols) cause wine spoilage in terms of smell and taste (“horse sweat”).

Agriculture:
Helpful when stables have to be cleaned, through atomization. In this case, bacteria and fungi, e.g. mold, are successfully attacked and, moreover, the composition according to the invention has a deodorant effect in that it kills smell-emitting germs.
Helpful when animal fur and feather treatment is carried out, through spray application. It may be employed to combat fungi (e.g. skin fungi or mold), bacteria, viruses and algae. When used for the purpose of tending furs and feathers it counteracts mites and skin fungi and enables small wounds to be cleaned and disinfected. Additionally, the inventive compositions have a deodorant effect in that they kill smell-emitting germs.
Helpful when hooves and claws of animals need care, through application of a solution. The composition acts against scurf and other diseases that may infest the hooves and claws of animals.
Helpful to protect bees against mites, fungi and bacteria which may infest bees and beehives. The inventive composition is atomized inside the beehive.

Furthermore, the composition according to the invention is suited for leather care, cleaning and disinfection of piping and hoses, especially for medical equipment and apparatus, the cleaning and disinfection of water reservoirs, for air and water sterilization, as a surface disinfectant and for swimming pool and whirlpool sterilization.

The composition may also be employed for the production of drugs and medicine, for example for active agents serving for cleaning and disinfection of wounds. Moreover, the composition in accordance with the invention effectively counteracts, inter alia, the SARS coronavirus, Bacillus anthracis, Escherichia coli, Klebsiella pneumoniae, Staphylococcus aureus, Pseudomonas aeruginosa, Streptococcus faecalis, Proteus mirabilis, Mycobacterium tuberculosis, Clostridium sporogenes, Candida albicans, house dust mites, varroa mites and many other causative organisms and parasites. Aside from this the composition may be used in treating athlete's foot disease and nail fungus or warts, as agent for mouth, teeth or skin care purposes. For example, it may be inhaled or applied to infested spots.

The inventive composition may be used as germicidal agent for the treatment of food, especially fruit and vegetables. In this case they counteract fungi, especially mold, bacteria, viruses, algae as well as eggs of fruit flies and other insects. As is necessary in each individual case, the food can be treated with the inventive composition by spraying or washing. Fruit and vegetables may also be briefly dipped into the inventive composition. Of particular advantage is the fact that the germicidal agent actually is inoffensive and entirely degradable by human beings and animals because they possess suitable enzymes causing percarboxylic acid to decompose, and the resultant carboxylic acid itself is virtually harmless. The same can be said for the composition when used as crop protection or plant fortification agent because although it effectively combats pests it will not cause damage to the plant itself. The invention may also beneficially be applied in the winegrowing sector, for instance in the treatment of grapevines or for the subsequent treatment of harvested grapes.

Applied at low concentration the inventive composition may also contribute to the sterilization of room air in that it combats airborne mold, bacteria, viruses and spores. For this purpose the respective solution may be used in air humidifiers or spraying apparatus producing an active agent mist covering rooms where its germicidal effect occurs quickly and safely. Moreover, distributing the active agent in the room air also counteracts unpleasant odors, for example of urine or tobacco. Cleaning the room air is particularly desirable in areas where many people come together so that there is an increased risk of illnesses spreading quickly. Examples in this case are hospitals, department stores, airports, railway stations and other public buildings and transportation means. Most helpful fields of use are seen for locations where air conditioning systems or room air odorizers are operated since the high air recirculation rates of such systems cause germs in the air to become rapidly distributed. The sterilization effect may be achieved in such a manner that the inventive composition is either added continuously or the systems are taken out of operation for a short period so that they can be sterilized by means of the composition proposed by the present invention. Especially if applied to room air odorizers the latter variant is preferred because in this way the scents/odorants used can also be oxidized by the percarboxylic acids.

The inventive composition may serve as a disinfectant for the treatment of surfaces. Applications in this case are, for instance, in the medical field where numerous appliances come into contact with disease-causing organisms, for example endoscopes or surgical instruments. On the other hand, the invention may also be applied in the industrial sector where the composition according to the invention may be used, for instance, in piping systems for disinfection purposes. This is especially important for applications in the food and beverage industry, e.g. in breweries, cideries, wineries and sparkling wine cellars, as well as fruit juice, lemonade or mineral water producing sectors.

In households the agent may primarily be used as a disinfectant, preferably in sanitary and kitchen areas. The composition is also well suited for sterilization of water and water containers, for example of water tanks in camping vehicles or boats. Likewise, the composition may be employed to sterilize swimming pools or whirlpools where chlorination hitherto customarily carried out for sterilization has caused significant, undesirable side effects.

The inventive composition may also be used for skin disinfection, a particularly helpful application in the medical field for hand cleaning purposes.

Aside from this, the invention also relates to the use of an ethoxylated, non-ethoxylated or oxypropylene units containing sorbitan ester for the purpose of stabilizing a composition containing at least one percarboxylic acid.

### EXAMPLE

1) An excess of hydrogen peroxide together with methanesulfonic acid is added to a solution comprising benzoic acid and acetic acid in polysorbate 20. Based on the total composition, the content at the start of the reaction amounts to 20% w/w polysorbate 20, 25% w/w benzoic acid, 5% w/w acetic acid, 38% w/w hydrogen peroxide as well as 2% w/w methanesulfonic acid, balance water. The reaction mixture is to be thoroughly mixed for approx. three weeks until equilibrium has been brought about. Finally, the solution is diluted with water to 10 times of its volume.

The product can be used as mold eliminator to combat mold and mold spores in a simple and safe manner. Mykotoxins will be deactivated as well.

2) Another composition (prior to conversion) comprises

150 g sorbic acid

250 g benzoic acid

100 ml polysorbate 20 (Tween 20)

1 ml H₂SO₄ (conc.)

4 ml H₃PO₄ (conc.)

to be topped up with H₂O₂ (35% w/w) and suspended and stirred at room temperature. The equilibrium has been established after approx. two weeks.

1-31. (canceled)

32. A composition containing at least one percarboxylic acid, wherein the composition further comprises at least one ethoxylated, non-ethoxylated or oxypropylene unit containing a sorbitan ester.

33. A composition according to claim 32, wherein the sorbitan ester is a polysorbate.

34. A composition according to claim 33, wherein the polysorbate is polysorbate 20, 21,40,60,61, 65, 80, 81 or 85.

35. A composition according to claim 32, wherein the concentration of the sorbitan ester ranges between 1 and 50% w/w.

36. A composition according to claim 35, wherein the concentration of the sorbitan ester ranges between 15 and 50% w/w.

37. A composition according to claim 36, wherein the concentration of the sorbitan ester ranges between 16 and 30% w/w.

38. A composition according to claim 37, wherein the concentration of the sorbitan ester ranges between 18 and 25% w/w.

39. A composition according to claim 32, wherein the concentration of the percarboxylic acid ranges between 0.1 and 45% w/w.

40. A composition according to claim 39, wherein the concentration of the percarboxylic acid ranges between 0.1 and 20% w/w.

41. A composition according to claim 40, wherein the concentration of the percarboxylic acid ranges between 5 and 15% w/w.

42. A composition according to claim 32, wherein the composition contains at least two percarboxylic acids.

43. A composition according to claim 32, wherein the composition contains at least one of an acid selected from the group consisting of perbenzoic acid, percitric acid, peracetic acid, persorbic acid, permalonic acid permaleic acid, perfumaric acid, pertartaric acid, and persuccinic acid.

44. A composition according to claim 43, wherein the composition contains perbenzoic acid and persorbic acid.

45. A composition according to claim 32, wherein the composition contains an inorganic peroxide.

46. A composition according to claim 45, wherein the inorganic peroxide is hydrogen peroxide.

47. A composition according to claim 32, wherein the composition contains a carboxylic acid corresponding to the percarboxylic acid.

48. A composition according to claim 32, wherein the composition contains at least one of solvents, surfactants, sequestrants, pH regulators, corrosion inhibitors, peracid stabilizers, complexing agents, defoaming agents, coloring substances or scents.

49. A composition according to claim 32, wherein the pH ranges between 0 and 6.

50. A method for producing a composition according to claim 32, comprising converting a carboxylic acid or a carboxylic acid derivative with an inorganic peroxide in the presence of an ethoxylated, non-ethoxylated or oxypropylene unit containing a sorbitan ester.

51. A method according to claim 50, wherein the inorganic peroxide is hydrogen peroxide.

52. A method according to claim 50, wherein the conversion takes place in an aqueous solution.

53. A method according to claim 50, further comprising diluting the composition with a solvent when the conversion has been completed.

54. A method according to claim 53, wherein the solvent is water.

55. A method according to claim 50, wherein the sorbitan ester is used at a concentration ranging between 16 and 30% w/w

56. A method according to claim 55, wherein the sorbitan ester is used at a concentration ranging between 18 and 25% w/w.

57. A method according to claim 50, wherein the inorganic peroxide is used at a concentration ranging between 20 and 40% w/w.

58. A method according to claim 57, wherein the inorganic peroxide is used at a concentration ranging between 30 and 40% w/w.

59. A method according to claim 50, wherein the conversion is effected in the presence of an acidic catalyst.

60. A method according to claim 59, wherein the acidic catalyst is phosphoric acid, sulfuric acid or methanesulfonic acid.

61. A method according to claim 50, wherein the conversion takes place during a period of between one day and six weeks.

62. A method according to claim 61, wherein the conversion takes place during a period of between two and four weeks.

63. A method according to claim 50, wherein an excess of inorganic peroxide is used compared to the carboxylic acid or carboxylic acid derivative.

64. A method of disinfecting, comprising using the composition according to claim 32 as a disinfectant.

65. A method of protecting or fortifying plants or crops, comprising using the composition according to claim 32 as a plant protection, crop protection, or plant fortification agent.

66. A method of killing germs, comprising using the composition according to claim 32 as a germicidal agent.

67. A method of killing at least one of bacteria, fungi, insects, or pests, comprising using the composition according to claim 32 as a bactericide, insecticide, or a pesticide.

68. A method of producing drugs, comprising using the composition according to claim 32 to produce drugs useful for treating bacterial or viral infectious diseases, mycoses, pediculoses, or for counteracting parasitic infestation.

69. A method of treating wine barrels, comprising using the composition according to claim 32 to treat the wine barrels.

70. A method of stabilizing a composition containing at least one percarboxylic acid, comprising using an ethoxylated, non-ethoxylated, or oxypropylene unit containing a sorbitan ester.
